(19) United States
(12) Patent Application Publication
(10) Pub. No.: US 2021/0391037 A1
(43) Pub. Date: Dec. 16, 2021

(54) SYSTEM AND METHOD FOR MOLECULAR DYNAMICS ANALYSIS

(71) Applicant: ENSCO, Inc., Springfield, VA (US)

(72) Inventors: Victor Francis LOTRICH, Gainesville, FL (US); Robert William MOLT, JR., Melbourne, FL (US)

(21) Appl. No.: 17/343,305

(22) Filed: Jun. 9, 2021

Related U.S. Application Data
(60) Provisional application No. 63/037,541, filed on Jun. 10, 2020.

(51) Int. Cl. G16C 20/30

(52) U.S. Cl. CPC G16C 20/30

(57) ABSTRACT

Aspects of the present disclosure describe systems and methods for molecular dynamics analysis of condensed matter and its transformations, including liquids, solids, aerosols, surfaces, and nanoparticles. In accordance with one embodiment, a computer system determines a force associated with a plurality of particles in a system, where the determining of the force is based on a potential energy function that comprises a first term proportional to the total charge of the system, and where the total charge is an integer. The computer system determines, based on the force, the position and velocity of a particle at a particular point in time, and an intermolecular property of the system is estimated based on the position and velocity.

### STATEMENT OF RELATED APPLICATIONS

The present application claims priority to, and incorporates fully by reference, U.S. Provisional Patent Application No. 63/037,541 filed Jun. 10, 2020.

### TECHNICAL FIELD

This disclosure relates generally to the determination and/or prediction of physical and chemical properties of condensed matter and its transformations, including liquids, solids, aerosols, surfaces, and nanoparticles. More particularly, it describes a system and method that is capable of predicting chemical and/or physical properties of such condensed matter including half-life, enthalpy of vaporization, boiling points, and molecular geometry, among others.

### BACKGROUND

There is an increasing realization that the chemical properties of particular chemical agents such as accidental industrial emissions, agricultural pesticides, toxic industrial chemicals (TICs), or toxic industrial materials (TIMs) etc., can be difficult to predict. Given that human exposure to such agents may pose significant health threats, a continuing need to understand their chemical properties—before such exposure—is of critical interest. Accordingly, systems and methods that can provide an accurate prediction of such chemical properties would represent a welcome addition to the art.

### SUMMARY

An advance in the art is made according to aspects of the present disclosure directed to systems and methods for molecular dynamics analysis of condensed matter and its transformations, including liquids, solids, aerosols, surfaces, and nanoparticles, thereby predicting chemical properties of potentially hazardous chemical agents including molecular geometry, IR spectra, chemical half-lives, enthalpy of vaporization, and boiling points.

In accordance with embodiments of the present disclosure, unique potential energy (PE) functions are employed that describe molecular interactions more accurately than is possible in the prior art. This enables the determining of chemical properties of condensed matter, without the necessity of a priori experimental knowledge, which is a capability completely lacking in the prior art. In addition, embodiments of the present disclosure provide an understanding of chemicals/materials that heretofore were experimentally difficult to perform, dangerous, and/or expensive to acquire. Importantly, the PE functions employed in embodiments of the present disclosure are physics-based, having direct, quantum mechanics meanings. In contrast, methods of the prior art employ PE functions that are chemistry-based and employ statistical correlations which are not experimentally observable.

### BRIEF DESCRIPTION OF THE DRAWINGS

A more complete understanding of the present disclosure may be realized by reference to the accompanying drawing in which:

FIG. 1 is a schematic diagram depicting multi-time step regions r and R, according to aspects of the present disclosure;

FIG. 2 depicts an illustrative flow diagram of aspects of a method for performing a molecular dynamics simulation, in accordance with one embodiment of the present disclosure.

FIG. 3 depicts an illustrative flow diagram of aspects of a method by which a molecular dynamics simulation uses mathematically general potential energy “basis functions,” matches a proper subset of specified basis functions, and produces mathematical quantities, in accordance with one embodiment of the present disclosure; and

FIG. 4 depicts a block diagram of an illustrative computer system operating in accordance with aspects and implementations of the present disclosure.

The illustrative embodiments are described more fully by the Figures and detailed description. Embodiments according to this disclosure may, however, be embodied in various forms and are not limited to specific or illustrative embodiments described in the drawing and detailed description.

### DESCRIPTION

The following merely illustrates the principles of the disclosure. It will thus be appreciated that those skilled in the art will be able to devise various arrangements which, although not explicitly described or shown herein, embody the principles of the disclosure and are included within its spirit and scope.

Furthermore, all examples and conditional language recited herein are intended to be only for pedagogical purposes to aid the reader in understanding the principles of the disclosure and the concepts contributed by the inventors to furthering the art and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that any block diagrams herein represent conceptual views of illustrative circuitry embodying the principles of the disclosure.

Unless otherwise explicitly specified herein, the FIGs. comprising the drawing are not drawn to scale.

We begin by noting once again that a continuing need exists for systems and methods that reliably predict the chemical properties of particular chemical materials including, pharmaceutical compounds, toxic industrial chemicals (TICS), toxic industrial materials (TIMs), agricultural compounds, and so forth. Because data pertaining to chemical and/or physical properties of such agents may be limited—particularly those agents that are novel and/or evolving—the ability to quantitatively, reliably, and accurately predict such properties is extremely desirable and may be of critical importance.

Advantageously, embodiments of the present disclosure employ intermolecular force considerations that significantly improve upon classical molecular dynamics approaches. More particularly, we employ a novel family of potential energy functions that exhibit a direct physical basis, and comprise fewer statistical parameters that are devoted entirely to data-fitting. The result is a superior estimation of intermolecular properties compared to the prior art.

By way of some further background, we note that there exist several drawbacks associated with potential energy functions used in prior-art biomolecular simulations. More particularly, such prior-art functions generally rely on “chemistry” rules of thumb and purely statistical fitting parameters. At present, classical potential energy functions in biomolecular simulation rely on chemistry intuition of what potentials roughly seem like and purely statistical fitting parameters.

Embodiments of the present disclosure are capable of remedying these deficiencies through the use of an intermolecular force theory representation of intermolecular interactions that comprises an electrical multipole expansion coupled to an exchange anti-symmetrization penalty [D. J. Griffiths, in Introd. to Quantum Mech., 1st ed. (Prentice Hall, Upper Saddle River, N.J., 1995), pp. 177-186, incorporated by reference as if set forth at length herein].

To better understand our approach, we first consider the manner in which interactions between covalent bonds are represented in the prior art—namely, using two harmonic oscillators and a Fourier term. Let N be the number of distinct atom pairs and let j be the index summing over these distinct interaction pairs. It should be noted that as used herein, the term “pair” does not necessarily refer to two atoms; rather, it refers to any minimum set of atoms that defines an interaction (e.g., two atoms for a covalent bond, three atoms for an angular configuration, four atoms for a dihedral, etc.).

Letting A, B, C, . . . denote specific individual atoms, the potential energy of intramolecular (or equivalently, “covalent”) interactions is typically defined in the prior art using an equation of the form:

E_{covalent}=½Σⱼ^{N}[k_{AB}(r_{AB}−r_{0,AB})²+k′_{ABC}(θ_{ABC}−θ_{0,ABC})²+k″_{ABCD}[1+(−1)^{j} cos(jω_{ABCD}+φ)]. Equation 1

where E_{covalent} denotes covalent potential energy totaled over all interaction pairs.

In Equation 1 above, k, k′, and k″ are parameterization constants, where capital Roman indices (in subscripts) distinguish how many nuclei are involved to specify a distinct parameter value. For example, k_{AB} is defined for every distinct possible pair of nuclei (e.g., A and B, etc.) for each combination of Roman indices. Variables r₀ and θ₀ refer to the “un-stretched” spring, and φ represents a similar energy reference point of zero, but within a Fourier-style expression that includes a Fourier term over every quadruplet of four nuclei (i.e., a dihedral angle). It should be noted that Equation 1 is intended to be expanded in a manner consistent with the many-body expansion (see reference 2); however for simplicity of notation and brevity, we leave Equation 1 in its current form.

This prior-art approach, typically employed by chemists, effectively performs a many-body expansion in the covalent energies over nuclei. Because the four-body energy interactions are more highly variable and are of smaller magnitude than two-body or three-body interactions, a Fourier series is a more logical choice than a Taylor series. The extent to which dihedral parameters are purely “fudge factors” in traditional dynamics simulations is well-known (see, e.g., Maier, J. A., Martinez, C., Kasavajhala, K., Wickstrom, L., Hauser, K. E., and Simmerling, C., J. Chem Theory Comput. 2015, 11, 3696), owing to the fact that there is no major interaction over the distances of four atoms. These fudge factors are typically employed to account for error or unanticipated circumstances, ensure a desired result, etc.

The potential energy of intramolecular (or equivalently, “non-covalent”) interactions is typically defined in the prior art using a Lennard-Jones potential and “Coulombic” potentials, using an equation of the form:

$${E}_{non \text{-} covalent} = {\sum }_{i j}^{N} [ {{ϵ}_{AB} ( \frac{{σ}_{A B}}{{r}_{ij}} )}^{12} - {( \frac{{σ}_{A B}}{{r}_{ij}} )}^{6} ] + \frac{{q}_{A} {q}_{B}}{{r}_{i j}} \qquad Equation 2$$

At first glance, the second term in Equation 2 above appears to be Coulomb's law,

$$( F = k \frac{{q}_{1} {q}_{2}}{{r}^{2}} ) ,$$

out it is not. Rather, chemists are assigning non-integer “partial charges” to q_{A} and q_{B} for species that are electrically neutral or genuinely charged, which is clearly not “physics-correct”. These partial charges assigned to atoms are known to be purely statistical fitting parameters, and often do not even correspond to chemistry intuition.

In accordance with aspects of the present disclosure, the “unphysical” qualities of Equation 2 are remedied by eliminating the Fourier term of Equation 1 and using a consistent multipole-based expansion of intermolecular interactions. Advantageously, such an approach leads to several unexpected benefits, which will be discussed vide infra. Hereafter, we omit the summations over all distinct pairs ij for simplicity of notation.

In one embodiment, the potential energy of intramolecular interactions and intermolecular interactions are defined, respectively, by Equations 3 and 4 below:

$$\begin{matrix}{E}_{covalent} = \frac{1}{2} [ {{k}_{A B} ( {r}_{A B} - {τ}_{0 , AB} )}^{2} + {k}_{A ; B C}^{′} ( {r}_{A B} - {r}_{0 , A B} ) ( {r}_{A C} - {R}_{0 , A C} ) + {k}_{B ; A C}^{′} ( {r}_{A B} - {r}_{0 , AB} ) ( {r}_{B C} - {r}_{0 , B C} ) + … ] & Equation 3 \\ {E}_{non \text{-} covalent} = {k}_{AB , 0} exp ( - {k}_{A B , 0}^{′} {r}_{AB} ) + {\sum }_{n = 1}^{N} \frac{{k}_{A B , n}}{{r}_{A B , n}} ( 1 - exp ( - {k}_{AB , n}^{′} {r}_{A B} ) ) & Equation 4\end{matrix}$$

These equations replace Equations 1 and 2 of the prior art.

The intramolecular parameters in Equation 3 are similar to those in Equation 1, aside from the fact that the cosine term has been omitted in Equation 3. In accordance with one embodiment, interactions over four nuclei are defined as being intermolecular (non-covalent), rather than intramolecular, and this intermolecular potential is better defined in the multipole moment expansion. In particular, the empirical softness of this dihedral potential is suggestive that it is better described as an intermolecular interaction.

In Equation 4, the first term represents the exchange-repulsion energy of fermions as an exponential. The second term summation represents the multipole expansion

$$( \frac{{k}_{AB , n}}{{r}_{A B}^{n}} )$$

multiplied by the exchange-repulsion, which is a decaying exponential. The exponentials in the multipole expansion represent the exchange-coupling to the terms in this electrical polarization expansion. This captures the extent to which the exchange modulates the multipole expansion, which represents the long-range electric behavior of the nuclei. The summation implicitly extends over all many-body expansion terms (doubles, triples, quadruples, etc.) [Elrod, M. J.; Saykally, R. J. Chem. Rev. 1994, 94 (7), 1975; Moszyński, R.; Cybulski, S. M.; Chalasiński, G. J. Chem. Phys. 1994, 100 (7), 4998; Bartlett, R. J.; Shavitt, I. Many-Body Methods in Chemistry and Physics; Cambridge University Press: Cambridge U.K., 2009; each of which is incorporated by reference as if set forth at length herein] as appropriate; pairwise additive terms are implemented. In order to facilitate understanding and simplify notation, we omit details of this implicit extension and instead focus on the fundamental differences between our potential energy functions and those of the prior art.

Each value of k is a constant for a given pair of nuclei (e.g., a particular oxygen atom and a particular hydrogen atom, etc.); these values are obtained prior to running a molecular dynamics simulation. The summation goes to N, where N serves as an accuracy control parameter. If one wishes to increase the accuracy of a simulation (e.g., when the observed accuracy is considered inadequate, etc.), one can increase the number of terms of in the summation (being aware that this may also increase the computational cost of the calculation), thereby providing systematic improvability to the calculations.

Advantages of our form of intermolecular potential (e.g., Equation 4 or variations thereof, etc.) compared to intermolecular approaches of the prior art (e.g., Equation 2 or variations thereof, etc.) are at least as follows:
Mapping to the MBPT(2)/aug′-cc-pVDZ, CCSD/aug′-cc-pVDZ, and CCSD(T)/aug′-cc-pVDZ solutions to the Schrödinger equation, which is of more consistent accuracy than current force fields.
Absence of Fourier soft potentials, which are, in practice, a set of fudge factors employed in the prior art. We instead utilize experimentally-derived observables, which are not subject to manipulation. Consequently, our results are physical, rather than statistical.
Proper asymptotic behavior of our expansion as distance increases, in contrast to functions of the prior art. In particular, our multipole expansion tends to zero as distance goes to infinity, whereas the Fourier expression of the prior art oscillates and never goes to zero as distance goes to infinity.
Our multipole expansion corresponds to measurable observables (e.g., C₆ dispersion coefficients, polarizabilities, etc.); this contrasts with the prior art, where the k values are purely data-fitting constants to minimize an error function, and have no empirical meaning related to experiments.
Our form allows explicit electrical polarizability terms, in contrast to the prior art;
Our form does not have any unphysical “Coulomb's Law” for non-integer charges, as is employed in the prior art. Accordingly, we are not relying on statistical cancellation of errors, as is done in the prior art.
Accuracy in our form can be systematically improved by adding more terms to the multipole summation. Thus, if one is willing to spend additional computational resources, one can get a more accurate answer. Prior-art approaches, in contrast, try to achieve greater accuracy by tweaking parameters.

Our improvements to the potential energy functions of the prior art enable the simulation of molecular dynamics (e.g., liquid-phase dynamics of DNA helices, etc.) with more reliable accuracy and greater flexibility. Moreover, because our force field is physically-based, we can track the contributions to the total energy function (i.e., do artificial DNA helix properties result from different solvent interactions, dispersion interactions, or both?). In contrast, force fields of the prior art have statistical parameters that can block the ability to interpret, physically, what forces contribute to what effects (due to, for example, the dihedral “fudge factors” and “charged” electrically neutral species).

In order to facilitate understanding of Equation 4, we write out the summation explicitly, and for simplicity consider only one nucleus pair A and B:

$${\sum }_{n = 1}^{N} \frac{{k}_{n}}{{r}_{n}} = \frac{{k}_{1}}{{r}^{1}} + \frac{{k}_{2}}{{r}^{2}} + \frac{{k}_{3}}{{r}^{3}} + \frac{{k}_{4}}{{r}^{4}} + \frac{{k}_{5}}{{r}^{5}} + \frac{{k}_{6}}{{r}^{6}} + … + \frac{{k}_{N}}{{r}^{n}} \qquad Equation 5$$

Equation 5 applies for every possible distinct pair of nuclei in the simulation (e.g., atoms A and B, atoms B and C, atoms C and D, atoms A and C, atoms A and D, atoms B and D, etc.). Each value of kₙ has a distinct physical meaning that may be measured experimentally. In classical electromagnetic theory, the above expansion corresponds to the multipole moment expansion. In quantum mechanics, all terms after r⁻⁵ also have a distinct meaning (dispersion) [Q. A. Smith, K. Ruedenberg, M. S. Gordon, and L. V. Slipchenko, J. Chem. Phys. 136, (2012), hereinafter “Smith”; A. Stone, in Theory Intermol. Forces, 2nd ed. (Oxford University Press, Oxford, U. K., 2013), pp. 64-68; A. D. Buckingham, Adv. Chem. Phys 12, 107 (1967); each of which is incorporated by reference as if set forth at length herein].

The first term in Equation 5, k₁/r¹, is known as an “electric monopole.” It can be shown that, in SI units, k₁ is proportional to the total charge of an electric system:

k₁∝Qₜₒₜₐₗ Equation 6
[Smith; D. J. Griffiths, in Introd. to Electrodyn., 4th ed. (Pearson, Glenview, 2013), pp. 151-158, incorporated by reference as if set forth at length herein].

The total charge Qₜₒₜₐₗ is a property of the whole system: the total number of negative and positive particles (all the protons plus electrons plus antimatter electrons plus pions plus tauons, etc. any charged particle). Qₜₒₜₐₗ is thus a physical observable that can be measured experimentally, not an adjustable (and non-integral) parameter as in the prior art. We emphasize this simple example as it is illustrative of the trends that continue in the aforementioned multipole moment expansion: each individual term in the expansion has a parameter which is an experimental observable, engendering physicality and self-consistency in a way the prior art does not.

For reasons which will become clear below, we momentarily skip over the second term k₂/r². The third term, k₃/r³, is related to an “electric dipole,” and it can be shown that, in SI units:

k₃∝−\vec{p}_{A}·\vec{p}_{B} Equation 7

where \vec{p} is the dipole vector of the atom/molecule in question, and A/B refer to the nucleus in question. For example, water has a net electric dipole moment, which is readily measured experimentally (or may be computed via some other means) and can be used to determine a value for k₃. Thus k₃, like k₁, is a physical constant, not an adjustable parameter.

Now returning to the second term in Equation 5, k₂/r² this term represents the interaction energy between the charge of the system (monopole) and the dipole discussed above with respect to third term, k₃/r³. For an electrically neutral system, k₁=k₂=0, since the total charge is zero.

The series in Equation 6 continues from dipole moments interacting (the r⁻³ term) to quadrupole moments interacting (the r⁻⁵ term) to octupole moments interacting (the r⁻⁷). In each case, an increasingly complicated set of experimental observables corresponds to k₃, k₅, and k₇, respectively. We can similarly have interactions between dipoles and quadrupoles (the r⁻⁴ term), quadrupoles and octupoles (r⁻⁸), etc. Starting with r⁻⁶, the terms in the series correspond to the sum of two individual effects: the classical electromagnetic multipole expansion described previously, and quantum mechanical dispersion effects.

The correspondences between constants k₁ through k₆ and their measurable experimental counterparts, as described above, are summarized in Table 1:

TABLE 1

| k value | Proportional to Measurable Experimental Quantity |
|---|---|
| k₁ | Total charge (monopole) interaction energy |
| k₂ | Monopole-Dipole interaction energy |
| k₃ | Dipole-Dipole interaction energy |
| k₄ | Dipole-Quadrupole interaction energy |
| k₅ | Quadrupole-Quadrupole interaction energy |
| k₆ | Sum of Dispersion and other electrical moments |

Documentation of the details of Table 1 may be found in either Griffiths, D. J. in Introduction to Electrodynamics, published by Pearson in Glenview, Ill. in 2013, pages 151-158 and Stone, A. in The Theory of Intermolecular Forces, published by Oxford University Press in Oxford, United Kingdom, 2013, pages 64-68; each of which is incorporated by reference as if set forth at length herein.

We now describe a computer-executable simulation (LAMDA) implementing our molecular dynamics approach. Advantageously, the simulation may be utilized to study all forms of condensed matter and its transformations, including liquids, solids, aerosols, surfaces, and nanoparticles.

The development of the LAMDA simulation was informed by our identification of several major flaws of the potential energy functions employed in the prior art:
The Fourier terms (dihedral energy functions) of the prior art require re-parameterization for every new study. In addition to being labor-intensive, it implies that these terms are statistical rather than physical in nature (i.e., if the Fourier terms were physical, they would not need to be adjusted).
The non-integer charge “Coulomb law” expression is clearly un-physical in much the same way, as noted above.
Given the statistical nature of prior-art potential energy functions, there is no systematic improvability. In other words, if the result provided by the potential energy function is not good enough (e.g., not sufficiently accurate, etc.), then the only way to possibly improve results is to return to re-parameterization and try again. In contrast, the math in embodiments of the present disclosure makes it clear how to get a more accurate result . . . and it is not by just re-parameterizing empirically.
The statistical nature of potential energy functions of the prior art results in continual “cancellation of errors”. In other words, scientists “hope” that the various flaws will compensate for one another (a little over-estimation, a little under-estimation).
Much of the prior art rests on chemistry intuition and legacy software code that favors simplicity over accuracy.

As is further described above. Equation 3 defines the molecular potential energy function internally, per molecule (or per “monomer”), and Equation 4 defines the molecular potential energy function externally, between molecule types. Accordingly, in one implementation, a first loop is employed for computed forces corresponding to monomer energy, and a second loop is employed for energy between each possible dimer (i.e., the intermolecular interactions are defined per dimer pair). This sharply contrasts with standard molecular dynamics (MD) force fields, which parameterize “charges” per monomer rather than per dimer.

Further, periodic boundary conditions and wall boundary conditions (i.e., an infinite potential “box”) may be implemented, with volume computed via a numeric mesh of boxes. If a particular molecule is within a box, then that box is counted as “populated”, such that that box's volume counts as “occupied.” A random number generator may then be used to assign an initial Gaussian distribution of velocities to molecules. The mean of the Gaussian distribution may be defined by the temperature with the variance set to 1 in atomic units (one Angstrom rather than one Bohr in our example implementation). In accordance with this example implementation, time is propagated via a Verlet algorithm (e.g., velocity Verlet, etc.).

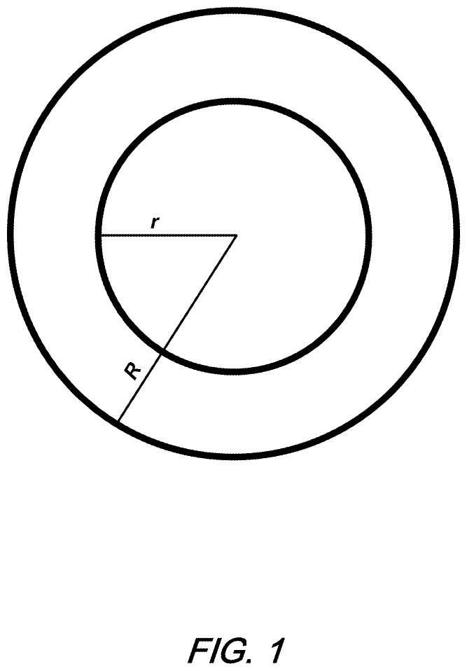

FIG. 1 is a schematic diagram depicting multi-time step regions r and R, according to aspects of the present disclosure. Region r refers to a short-range interaction, for which smaller time steps are used in the simulation (for example ˜1-2fs time steps), and region R refers to a long-range interaction, for which larger time steps are used (e.g., 2fs to 4s, etc.). In one implementation of the simulation, each region has its own pair-list of nearest neighbors, and the values of r and R defining the short- and long-range regions can be set by a user and provided as input.

In accordance with one embodiment, Equation 4 is modified for long-range regions. Recall that Equation 4 has a multipole expansion multiplied by an exponential representing the exchange. In the long-range interaction region, we force the exponential argument to infinity, thereby eliminating any exponential dependence. As a result, only multipole terms remain, and there is no need to compute the exponential, saving computation time. It should be noted that this is not a physical approximation, as the exchange term is already negligible in this region. In the short-range interaction zone, no modification is made to the potential.

During execution, forces are computed per time step based on a list of molecule pair types. Each distinct molecule pair's force is computed from an array with N+1 elements, where N is the variable in Equation 4 representing the number of terms in the summation. An algorithm is executed to reduce the dimensionality of the force array commensurate with the number of zeroes in the array. For example, a strictly Lennard-Jones potential may be reduced from [0,0,0,0,0,0,0,0,0,0,0,0,0] to [C₆,C₁₂] by an array contraction routine to prevent needless computation of zeroes. This can help compensate for the potentially-large number of terms in the summation of Equation 4 (i.e., when using a large value of N).

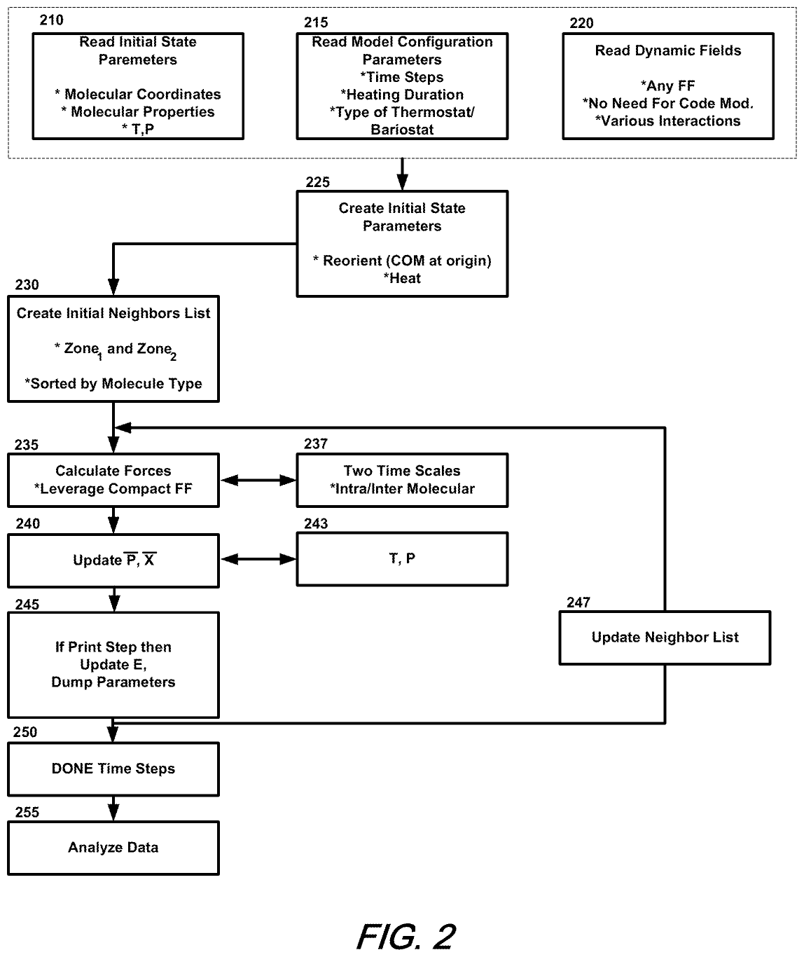

FIG. 2 depicts an illustrative flow diagram of aspects of a method for performing a molecular dynamics simulation, in accordance with one embodiment of the present disclosure. The method may be performed by processing logic comprising hardware (circuitry, dedicated logic, etc.), software (such as is run on a general purpose computer system or a dedicated machine), or a combination of both. It should be noted that in some implementations, one or more blocks depicted in FIG. 2 might be performed simultaneously, or in a different order than that depicted. In addition, while a single execution of the method is depicted in FIG. 2, the method may be performed multiple times (e.g., a pre-determined number of times, an infinite loop, etc.).

At Block 210, the material state of interest is provided as input (e.g., what atoms, what their relative distances and orientations, the temperature of the system, the pressure of the system, etc.).

At Block 215, the mathematics that set bounds on how time, temperature, pressure, etc. vary in the simulation is provided as input. This might include, for example, specification of a time period of 100 ns, time steps of 2fs, a Nose-Hoover type thermostat (see, e.g., Nose, S. J. Chem. Phys. 1984, 81 (1), 511.), a Berendsen type barostat (see, e.g., Berendsen, H. J. C.; Postma, J. P. M.; van Gunsteren, W. F.; DiNola, A.; Haak, J. R. J. Chem. Phys. 1984, 81, 3684; each of which is incorporated by reference as if set forth at length herein), etc.

At Block 220, one or more dynamic fields and/or one or more potential energy functions are provided as input.

With these inputs provided, one may now create the beginning of the simulation, assigning positions, velocities, and potential energy functions. Such “initial state” parameters are created as indicated by Block 225.

At Block 230, a “neighbors” list is created. The neighbors list assigns zones of “closeness”. More particularly, if atoms are sufficiently close, more forces are computed, because greater numerical care is warranted. If atoms are further apart, then the forces acting are weaker, and less numerical care may be necessary—resulting in greater computational efficiency. The choice(s) with respect to how close atoms must be to be considered in different zones is governed by input files (Block 215).

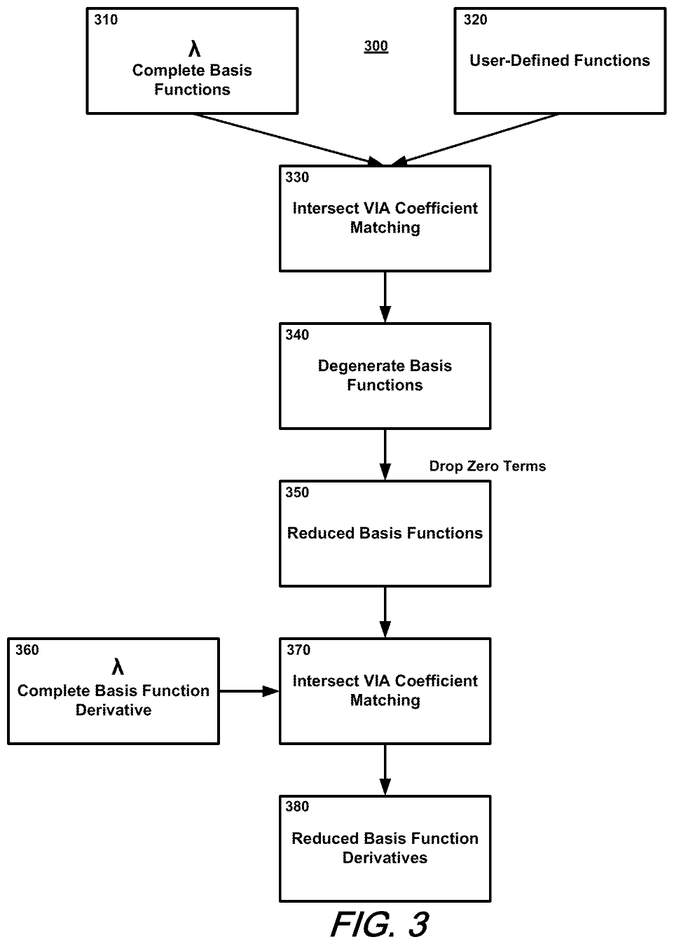

At this point, the “heart” of the simulation is entered, namely, a step-by-step time simulation which feeds back into itself until the total time steps have finished. At Block 235, forces (derivatives of the energy functions) are computed in the compact FF (compactness is described in detail below with respect to FIG. 3). This computation interacts with a module associated with the two time scales of the process at Block 237. Note that just as we defined two interacting zones for the forces, there are two interacting time-scales [multi-time-step molecular dynamics—see, e.g., Morrone, J. A.; Zhou, R.; Berne, B. J. J. Chem. Theory Comput. 2010, 6 (6), 1798; and Tuckerman, M.; Berne, B. J.; Martyna, G. J. J. Chem. Phys. 1992, 97 (3), 1990; each of which is incorporated by reference as if set forth at length herein], one for intramolecular forces and one for intermolecular forces. When the force fields are initialized (Block 225), this distinction is made. As will be appreciated by those skilled in the art, larger time steps are viable for the intermolecular forces as opposed to intramolecular forces.

At Block 240, mean pressure and position of the system are updated. This interacts in defining the new temperature and pressure (i.e., barostat and thermostat) at Block 243. If the print step flag is on (Block 245), then print is written to file to record energy, temperature, etc. As will be appreciated by those skilled in the art, in some embodiments it may not be important to print everything, as writing to disk can be time-intensive [see, e.g., Götz, A. W.; Williamson, M. J.; Xu, D.; Poole, D.; Le Grand, S.; Walker, R. C. J. Chem. Theory Comput. 2012, 8, 1542; each of which is incorporated by reference as if set forth at length herein] and can negatively affect performance in terms of total time simulated. At Block 247, the list of nearest neighbors is updated, as the positions have been revised, and therefore the numerical accuracy required of different forces may change.

When the final time step has been reached at Block 250, the simulation ends, and one may analyze the output file data at Block 255. In one embodiment, the output includes time snapshots of each particle's position and velocity (e.g., the position/velocity of a first particle at a first time t₁, the position/velocity of the first particle at a second time t₂, the position/velocity of a second particle at the first time t₁, the position/velocity of the second particle at the second time t₂, etc.).

At this point, one or more intermolecular properties of the system can be estimated based on the position/velocity data. In some instances, the estimation may be based solely on position/velocity data, while in some other instances the estimation may be based on other output data as well.

Intermolecular properties may include enthalpy of vaporization, one or more half-lives, one or more boiling points, one or more IR spectra, molecular geometry, and so forth. In some instances, an intermolecular property may be a particular numeric value (e.g., a boiling point of 100 degrees Celsius, etc.), while in some other instances, an intermolecular property may be expressed with respect to a threshold (e.g., a boiling point of at least 100 degrees Celsius, etc.), while in still other instances, an intermolecular property may be expressed as another type of relationship (e.g., boiling point of matter X exceeding the boiling point of matter Y, etc.). In yet other instances, an intermolecular property may be qualitative in nature (e.g., the phase of a material [solid vs. liquid vs. plasma], etc.).

FIG. 3 depicts an illustrative flow diagram of aspects of a method by which a molecular dynamics simulation uses mathematically general potential energy “basis functions,” matches a proper subset of specified basis functions, and produces mathematical quantities in a computationally-efficient manner, in accordance with one embodiment of the present disclosure. The method may be performed by processing logic comprising hardware (circuitry, dedicated logic, etc.), software (such as is run on a general purpose computer system or a dedicated machine), or a combination of both. It should be noted that in some implementations, one or more blocks depicted in FIG. 3 might be performed simultaneously, or in a different order than that depicted. In addition, while a single execution of the method is depicted in FIG. 3, the method may be performed multiple times (e.g., a pre-determined number of times, an infinite loop, etc.).

At Block 310, a set of one or more complete basis functions is defined/generated [see, e.g. Griffiths, D. J. In Introduction to Quantum Mechanics; Prentice Hall: Upper Saddle River, N.J., 1995; pp 27-29; and Bender, C. M.; Orszag, S. A. In Advanced Mathematical Methods for Scientists and Engineers I; Springer: New York, 1999; p 351; each of which is incorporated by reference as if set forth at length herein], and at Block 320, a user specifies a particular desired mathematical form of the potential energy. Typically this mathematical form, which we refer to as g(x), is a finite number of functions, given computational implementation.

In one embodiment, the set of complete basis functions includes a radial expression of the multipole expansion weighted by an exponential decay represented by:

$${\sum }_{n = 0}^{∞} \frac{{a}_{n} {e}^{- {b}_{{n}^{r}}}}{{r}^{n}} . \qquad Equation 8$$

This particular basis expansion is complete by virtue of the fact that spherical harmonics are complete, and the multipole expansion itself may be expressed in terms of spherical harmonics [see, e.g., Arfken, G. Mathematical Methods for Physicists; Academic Press: New York, 1970, incorporated by reference as if set forth at length herein]. Consequently, it is always possible to select a set of aₙ such that:

$$g ( x ) = {\sum }_{n = 0}^{∞} \frac{{a}_{n} {e}^{- {b}_{n} r}}{{r}^{n}} \qquad Equation 9$$

and we may thus express any user-specified function (e.g., specified by the user at Block 320, etc.) in terms of one or more of the basis functions defined/generated at Block 310. (provided that there are a finite number of discontinuities in g(X)). Thus, we can represent any function, mathematically, in the above formalism, and then get rid of any extra unneeded functions to make the summation of Equation 9 finite rather than infinite. In one example implementation, the expansion is carried out to n=14 for finite implementation of the multipole expansion. This can be easily expanded to whatever value is desired.

At Block 330, a user can engage in coefficient matching between user-defined function(s) g(x) and our complete basis functions. Specifically, the user can designate (e.g., in an input file, etc.) values of aₙ that produce g(x). This provides a set of degenerate basis function(s) at Block 340, in that there will be at least one value for which aₙ=0.

At Block 350, the degenerate basis function(s) are dropped to a result in an orthonormal set of “reduced” basis functions. This avoids performing mathematical operations on needless basis functions, thereby improving computational efficiency.

At Block 360, the derivatives of the basis functions are computed, which will be used by a molecular dynamics engine (implicitly between Block 350 and Block 360). At Block 380, the same process of coefficient matching is repeated between the complete basis functions and the reduced basis function derivatives.

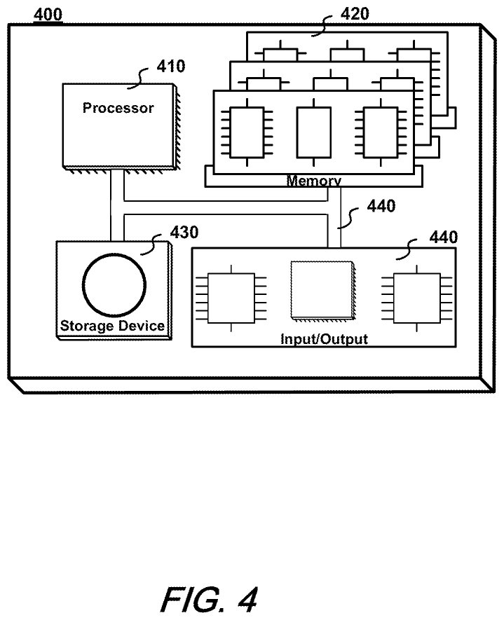

FIG. 4 depicts a block diagram of an illustrative computer system 400 operating in accordance with aspects and implementations of the present disclosure. The above-described methods of the present disclosure may be implemented on the computer system 400 as stored program control instructions.

As shown in FIG. 4, computer system 400 comprises processor 410, memory 420, storage device 430, and input/output structure(s) 440. One or more input/output devices may include a display having any of a variety of known user interfaces including graphical user interfaces (not specifically shown). One or more busses 450 typically interconnect the components, 410, 420, 430, and 440. Processor 410 may be a single or multi core.

Processor 410 is capable of executing instructions for performing one or more steps described in FIG. 2 and/or FIG. 3. Such instructions may be stored in memory 420 or storage device 430, or may be stored remotely and accessed via one or more input/output mechanisms. Data and/or information may be received and output using one or more input/output devices.

Memory 420 is capable of storing data and may be a computer-readable medium, such as volatile or non-volatile memory. Storage device 430 may be a flash memory device, a disk drive, an optical disk device, a tape device employing magnetic, optical, or other recording technologies, etc. that is capable of providing storage for system 400, including for example, the previously described methods.

Input/output structures 440 are capable of providing input/output operations for system 400. Input/output devices utilizing these structures may include, for example, keyboards, displays, pointing devices, microphones, and network interface adaptors including wired and/or wireless ones. As shown and may be readily appreciated by those skilled in the art, computer system 400 for use with the present disclosure may be implemented in a desktop computer package 460, a laptop computer 470, a hand-held computer, for example a tablet computer, personal digital assistant or Smartphone, or one or more server computers which may advantageously comprise or be part of a “cloud” computer and attendant services provided therefrom.

While we have presented this disclosure using some specific examples, those skilled in the art will recognize that our teachings are not so limited. Accordingly, this disclosure should be only limited by the scope of the claims attached hereto.

1. A method comprising:
determining, by a computer system, a force associated with a plurality of particles in a system, wherein the determining is based on a potential energy function that comprises a first term proportional to the total charge of the system, and wherein the total charge is an integer;
determining, based on the force, (i) a first position of a first particle of the plurality of particles at a first time t₁, and (ii) a first velocity of the first particle at time t₁; and
estimating, based on the first position and the first velocity, an intermolecular property of the system.

2. The method of claim 1 wherein the total charge is measured experimentally.

3. The method of claim 1 wherein the potential energy function further comprises a second term that is proportional to a dot product of (a) a dipole vector of the first particle, and (b) a dipole vector of a second particle of the plurality of particles.

4. The method of claim 1 wherein the determining of the force is further based on one or both of a temperature or a pressure.

5. The method of claim 1 further comprising determining, based on the force, (iii) a second position of the first particle at a second time t₂, and (iv) a second velocity of the first particle at time t₂.

6. The method of claim 1 further comprising determining, based on the force, (iii) a position of a second particle of the plurality of particles at time t₁, and (ii) a velocity of the second particle at time t₁.

7. The method of claim 7 wherein the potential energy function lacks a Fourier term.

8. A method comprising:
determining, by a computer system, a first force associated with a plurality of particles in a system, wherein the determining is based on a potential energy function comprising a multipole expansion;
determining, based on the first force, (i) a first position of a first particle of the plurality of particles at a first time t₁, and (ii) a first velocity of the first particle at time t₁; and
estimating, based on the first position and the first velocity, an intermolecular property of the system.

9. The method of claim 8 wherein the multipole expansion comprises a first term that is based on a first measurable experimental quantity and a second term that is based on a second measurable experimental quantity.

10. The method of claim 8 wherein the multipole expansion is coupled to an exchange anti-symmetrization penalty.

11. The method of claim 8 wherein the potential energy function lacks a Fourier term.

12. The method of claim 8 further comprising determining a second force associated with the plurality of particles, wherein the determining of the first position and the first velocity are further based on the second force.

13. The method of claim 8 wherein the potential energy function comprises a term whose value is based one or more quantum mechanical dispersion effects.

14. A method comprising:
determining, by a computer system, a force based on an energy associated with a dimer pair of particles in a system;
determining, based on the force, (i) a first position of a first particle of the plurality of particles at a first time t₁, and (ii) a first velocity of the first particle at time t₁; and
estimating, based on the first position and the first velocity, an intermolecular property of the system.

15. The method of claim 14 wherein the determining of the force is further based on an energy associated with a monomer particle.

16. The method of claim 14 wherein the force is computed using a complete basis function.

17. A method comprising:
executing, by a computer system, a molecular dynamics simulation of a system comprising a plurality of particles, the simulation using a first time step for a first molecular interaction and a second time step for a second molecular interaction during a single execution of the simulation, and the second time step different than the first time step;
obtaining, from output generated by the executing of the simulation, (i) a first position of a first particle of the plurality of particles at a first time t₁, and (ii) a first velocity of the first particle at time t₁; and
estimating, based on the first position and the first velocity, an intermolecular property of the system.

18. The method of claim 17 wherein the first molecular interaction is at a shorter range than the second molecular interaction, and wherein the first time step is smaller than the second time step.

19. The method of claim 17 wherein the executing of the simulation comprises computing a derivative based on one or both of a complete basis function and a function specified by a user of the simulation.

20. The method of claim 17 wherein the executing of the simulation comprises reducing the dimensionality of a force array.
